(19) United States
(12) Patent Application Publication
(10) Pub. No.: US 2018/0271473 A1
(43) Pub. Date: Sep. 27, 2018

(54) CALIBRATION OF QUANTITATIVE BIOMARKER IMAGING

(71) Applicant: KONINKLIJKE PHILIPS N.V., EINDHOVEN (NL)

(72) Inventor: Raz CARMI, HAIFA (IL)

(21) Appl. No.: 15/542,465

(22) PCT Filed: Jan. 7, 2016
(86) PCT No.: PCT/IB2016/050053

§ 371 Date: Jul. 10, 2017

Related U.S. Application Data
(60) Provisional application No. 62/104,880, filed on Jan. 19, 2015.

(51) Int. Cl. A61B 6/00; G16H 50/70; A61B 6/03

(52) U.S. Cl. CPC A61B 6/582; A61B 6/5211; A61B 6/032; G16H 50/70

(57) ABSTRACT

A quantitative measurement system includes a quantitative imaging biomarker calibrator (116). The quantitative imaging biomarker calibrator (116) receives one or more pre-calibrated quantitative measurements of imaging data obtained according to a global features analysis (230) and a class-combination (232) of a biological target, an indicated biology, an imaging acquisition modality and a protocol, a data processing technique, and a contrast agent. The quantitative imaging biomarker calibrator (116) applies an identified function (234) to the one or more pre-calibrated quantitative measurements to compute the one or more calibrated quantitative measurements based on a target class-combination (236) which is different from the class-combination (232).

### FIELD OF THE INVENTION

The following generally relates to medical informatics and quantitative imaging using biomarkers, and is described with particular application to standardization of quantified biomarkers across different imaging procedures.

### BACKGROUND OF THE INVENTION

As defined by an initiative of the Radiological Society of North America, quantitative imaging is the extraction of quantifiable features from medical images for the assessment of normal or the severity, degree of change, or status of a disease, injury, or chronic condition relative to normal. Such features are usually defined as imaging biomarkers. Biomarkers are used to target anatomical, functional and/or molecular features in a subject and enable quantifiable measurement in imaging. The distribution and measurement of anatomical, functional and/or molecular features based on a biomarker is affected by imaging modalities and imaging parameters, imaging agent(s), and data processing algorithms employed.

Representative examples of medical imaging biomarkers are blood perfusion and flow characteristics measured using contrast agent, uptake of radiotracers in abnormal tissues, level of abnormal proteins aggregation in the brain, degree of the severity of artery occlusion by plaque, myocardium functionality parameters, and structure and texture patterns of diseased lung tissues.

Biomarker assessment can be an indirect result of measured features in a subject and can be based on additional general models and assumptions. For example, the biomarkers measure blood perfusion and blood permeability are indirect measures of the imaging of dynamic changes of administered contrast agent to the subject.

Focus in quantitative imaging is given to repeatable imaging processes in research and patient care, and standardization of individual procedures to produce verifiable results. For example, in research studies, the imaging modality and parameters, contrast agent and concentrations, and description of data processing are typically included in a reported study. Imaging of patients typically include protocols that specify the imaging modality and parameters, contrast agent, and use of certain data processing algorithms or software implicitly or explicitly. Choice of a particular protocol includes tradeoffs and can include healthcare practitioner and/or healthcare organization preferences. Appropriate methods to assess a desirable biomarker vary in their imaging attributes of accuracy, sensitivity, and specificity, and selection of a particular method involves consideration of each attribute. For example, positron emission tomography (PET) can use ¹¹C-PM, ¹⁸F-Florbetapir or ¹⁸F-Flutenmetamol, each approved for imaging of Beta-Amyloid plaque in Alzheimer disease assessment, and with varying attributes and cost. Imaging modalities are constantly being updated with new parameters and capabilities. New contrast agents are developed, and new techniques are added for processing image data, measuring biomarkers, and quantifying features.

As change occurs, the healthcare practitioner or researcher is faced with comparing current results with a previous result, a normal result, or determining a severity, where the comparison involves different imaging modalities and/or parameters, different contrast agents and/or different processing algorithms. For example, blood flow can be measured indirectly using dynamic contrast enhanced computed tomography (CT) with an iodine contrast agent, dynamic contrast enhanced magnetic resonance imaging (MM) with a gadolinium contrast agent, or dynamic positron emission tomography (PET) with a ¹⁸F-FDG or a ¹⁵O—H₂O contrast agent. The healthcare practitioner may be faced with comparing current results of a patient from the dynamic contrast enhanced CT with the iodine contrast agent with prior results from another healthcare provider performed with the dynamic PET with ¹⁸F-FDG to determine whether a tumor has changed in vascularization between treatments. The researcher may be constrained in a radiation therapy study to using dynamic contrast enhanced MRI with the gadolinium and want to benchmark results of that study with another comparable study reported with dynamic PET with the ¹⁵O—H₂O contrast agent.

As quantitative imaging evolves, particular techniques emerge, normative or formal standards form and/or change, changing from one technique to another typically lags in organizations due to cultural and/or monetary pressures. Sometimes choices continue to exist when no one technique establishes preeminence. However, healthcare practitioners and/or researchers are faced with unclear comparisons across different techniques to make decisions about healthcare choices and/or value of treatments.

### SUMMARY OF THE INVENTION

Aspects described herein address the above-referenced problems and others.

The following describes an approach for calibrating quantitative biomarker imaging. A multi-class calibration database is implemented and provides calibration of quantitative measurements between different classes of imaging procedures. Classes of imaging procedures can vary by biological target, indicated biology, imaging modality and protocol, data processing algorithm, and/or contrast agent. The calibration is implemented for an individual imaging procedure to transform non-calibrated quantitative measurements to calibrated quantitative measurements for the assessment of normal or the severity, degree of change, or status of a disease, injury, or chronic condition relative to normal.

In one aspect, a quantitative measurement system includes a quantitative imaging biomarker calibrator. The quantitative imaging biomarker calibrator receives one or more pre-calibrated quantitative measurements of imaging data obtained according to a global features analysis and a class-combination of a biological target, an indicated biology, an imaging acquisition modality and a protocol, a data processing technique, and a contrast agent. The quantitative imaging biomarker calibrator applies an identified function to the one or more pre-calibrated quantitative measurements to compute the one or more calibrated quantitative measurements based on a target class-combination, which is different from the class-combination.

In another aspect, a method of quantitative measurement includes applying an identified function to one or more pre-calibrated quantitative measurements obtained according to a global features analysis and a class-combination of a biological target, an indicated biology, an imaging acquisition modality and a protocol, a data processing technique, and a contrast agent to compute the one or more calibrated quantitative measurements based on a target class-combination, which is different from the class-combination.

In another aspect, a quantitative measurement system includes a multi-class calibration database, a global feature analyzer and a quantitative imaging biomarker calibrator. The multi-class calibration database includes a plurality of global feature analysis and class combinations of a biological target, an indicated biology, an imaging acquisition modality and method, a data processing algorithm, and a contrast agent, and each class combination includes at least one function. The global feature analyzer receives a selection of a global features analysis for imaging data and class information to identify at least one function in the multi-class calibration database which transforms one or more quantitative measurements of the imaging data to one or more calibrated quantitative measurements. The quantitative imaging biomarker calibrator applies the identified at least one function to the one or more quantitative measurements of the imaging data to compute the one or more calibrated quantitative measurements. The identified at least one function of the identified class combination transforms the one or more quantitative measurements of the imaging data to the one or more calibrated quantified measurements of a target class combination. The target class combination is different from the identified class combination.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates an example cross procedure quantitative biomarker imaging system.

FIGS. 2A and 2B show partial examples of multi-class calibration data from a multi-class calibration database.

FIG. 3 flowcharts an example method of cross procedure quantitative biomarker imaging.

FIG. 4 flowcharts another example method of cross procedure quantitative biomarker imaging.

### DETAILED DESCRIPTION OF EMBODIMENTS

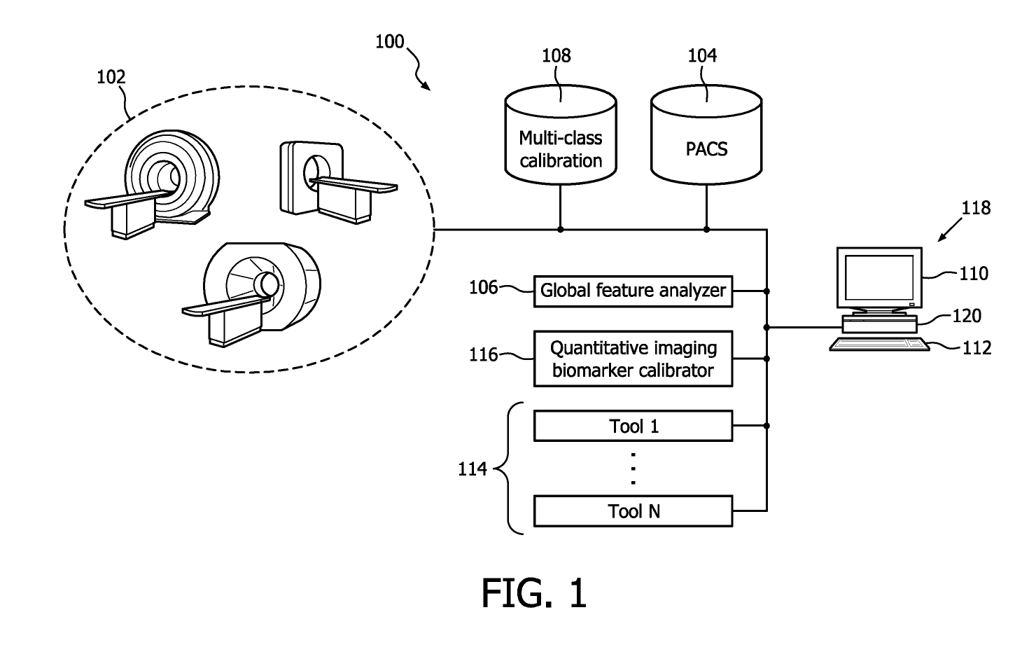

Initially referring to FIG. 1, an example cross procedure quantitative biomarker imaging system 100 is schematically illustrated. Imaging data is generated by one or more imaging devices 102, such as a CT scanner, a PET scanner, an MRI scanner, a SPECT scanner, an ultrasound (US) scanner, a hybrid, a combination, and the like. The imaging data is generated based on one or more modalities using a protocol. The protocol can be used to set and adjust the imaging system, the administered material, the patient conditioning, etc. The imaging data is of a subject or object, such as a region of interest of a patient. The imaging data includes a contrast agent or a tracer material. The description herein uses the notation contrast agent to indicate various options of materials used in medical imaging such as radiopaque materials, magnetic or radiofrequency responding materials, radioactive tracers, optical florescence tracers, ultrasonic microbubble tracers and others. The imaging data can include descriptive information about the source of the imaging data, such as Digital Imaging and Communications in Medicine (DICOM) standard metadata. The imaging data can be stored in a system or computer memory, such as a Picture Archiving and Communication System (PACS) 104, Vendor Neutral Archive (VNA), Radiological Information System (RIS), Hospital Information System (HIS), Electronic Medical Record (EMR), and the like.

A global feature analyzer 106 receives the imaging data from the PACS 104 or the imaging device 102. The global feature analyzer 106 receives a global feature analysis to be performed on the imaging data. The global feature analysis can retrieve a list of global feature analysis from a multi-class calibration database and present the retrieved list on a display device 110. The list can be refined based on the received imaging data. For example, using the metadata from the imaging data, one or more of the biological target, indicated biology, imaging acquisition modality and/or protocol, data processing algorithm, or the contrast agent are automatically identified. In one instance, inputs or signals received from one or more input devices 112, such as a mouse, keyboard, microphone, touch screen, and the like, which indicate the global feature analysis. In one instance, a combination of inputs and metadata refines the list and/or inputs the selected global feature analysis.

The global feature analyzer 106 invokes one or more tools 114 to quantitatively analyze the imaging data using known data processing techniques to generate one or more quantitative measurements of the imaging data. In one instance, the tools 114 are manually invoked using the input device 112. For example, a first tool segments and generates a volumetric map, e.g. spatial structure with biomarker presence by voxel, of a region of interest, and a second tool computes one or more quantitative measures of the biomarker present in the volumetric map, such as a mean, median, minimum, maximum, variance, etc. of concentration of the biomarker in a structure defined by the volumetric map. The one or more computed quantitative measures of the biomarker are specific pre-calibration measurements according to the imaging device and the protocol, the biological target, the indicated biology, the data processing algorithm, and the contrast agent.

A quantitative imaging biomarker calibrator 116 retrieves a calibration function from the multi-class calibration database 108 based on the received global features analysis and the combination of the imaging device and the protocol, the biological target, the indicated biology, the data processing algorithm, and the contrast agent. The calibrator 116 applies the function to the computed quantitative measures, which are pre-calibrated measures, to compute one or more calibrated measures. For example, a mean concentration of Beta-Amyloid plaque in brain grey matter using a PET modality as measured with an ¹¹C-PIB contrast agent is computed from a mean concentration of Beta-Amyloid plaque in brain grey matter as measured from the imaging data using a PET modality with an ¹⁸F-Florbetapir contrast agent. The computed calibrated measures are displayed on the display device 110. The computed pre-calibrated measures can additionally be displayed on the display device 110.

The calibration functions are based on comparative studies between the different imaging modalities and protocols, contrast agents, and data processing techniques, such as public or published clinical trials, or clinical trial data. The calibration functions can include various statistics, such as minimum, maximum, mean, median, standard deviation, and the like. The calibration functions can include visualization and/or imaging manipulation functions which map the imaging data and/or derived portions from a pre-calibrated imaging space to a calibrated space, e.g. transform the imaging data from representation in one protocol to another protocol, from one imaging modality to another imaging modality and/or one data processing technique to another. The calibration function transforms the pre-calibration measurements of a class-combination to calibrated measurements of a target class-combination which is different by at least one instance of one class. For example, a contrast agent is different between the class-combination and the target class-combination, an imaging protocol is different between the class-combination and the target class-combination, a data processing technique is different between the class-combination and the target class-combination.

The calibration function can be expressed as a single or multiple linear function of a form y=a₁*x_{1+ . . . +}aₙxₙ+b, where a_{1 . . .} aₙ, and b are constants, and x_{1 . . .} xₙ are pre-calibrated quantitative measures and y is a calibrated measure. The calibration function can be expressed as a non-linear function of a form y=f(x), where x is a pre-calibrated measure, f is a non-linear function, and y is the calibrated measure. The calibration function can be of a general form [y₁, y₂, . . . ]=f2[x₁, x₂, . . . ], where yᵢ is a calibrated measure and xⱼ is a pre-calibrated measure. The calibration functions can be revised and updated based on new data and/or standards emerge or change.

In one instance, the calibrated measurements can be derived and/or operated with results from computed aided detection software, e.g. detects lesions based on biomarkers. In another instance, the calibrated measurements can be derived and/or operated with results from existing biomarker applications, such as tumor tracking, lesion and module assessment, plaque distribution assessment, and the like.

The display device 110 and/or input device 112 can comprise a computing device 118, such as a desktop computer, laptop computer, tablet computer, smartphone, body worn computing device, and the like. The computing device 118 includes one or more data processors 120, such as an electronic data processor, digital processor, optical processor, microprocessor, and the like. The computing device 118 can include a distributed computer configuration such as a client computer and a server computer communicatively connected, a peer computer communicatively connected to another peer computer, and the like.

The global feature analyzer 106 and the calibrator 116 are suitably embodied by a data processor configured to execute computer readable instructions stored in a non-transitory computer readable storage medium or computer readable memory, e.g. software, such as the data processor 120 of the computing device 118. The disclosed global feature analysis and calibration techniques are suitably implemented using a non-transitory storage medium storing instructions readable by the data processing device and executable by the data processing device to perform the disclosed techniques. The data processor 120 can also execute computer readable instructions carried by a carrier wave, a signal or other transitory medium to perform the disclosed techniques.

The multi-class calibration database 108 can include file organization, database management structures, such as object and/or element definition and organization, data structures, and the like. The multi-class calibration database 108 can include computer memory or storage mediums both transitory and non-transitory. The multi-class calibration database 108 can include storage mediums, such as local or remote storage, hard disk, solid state memory, cloud storage, and the like.

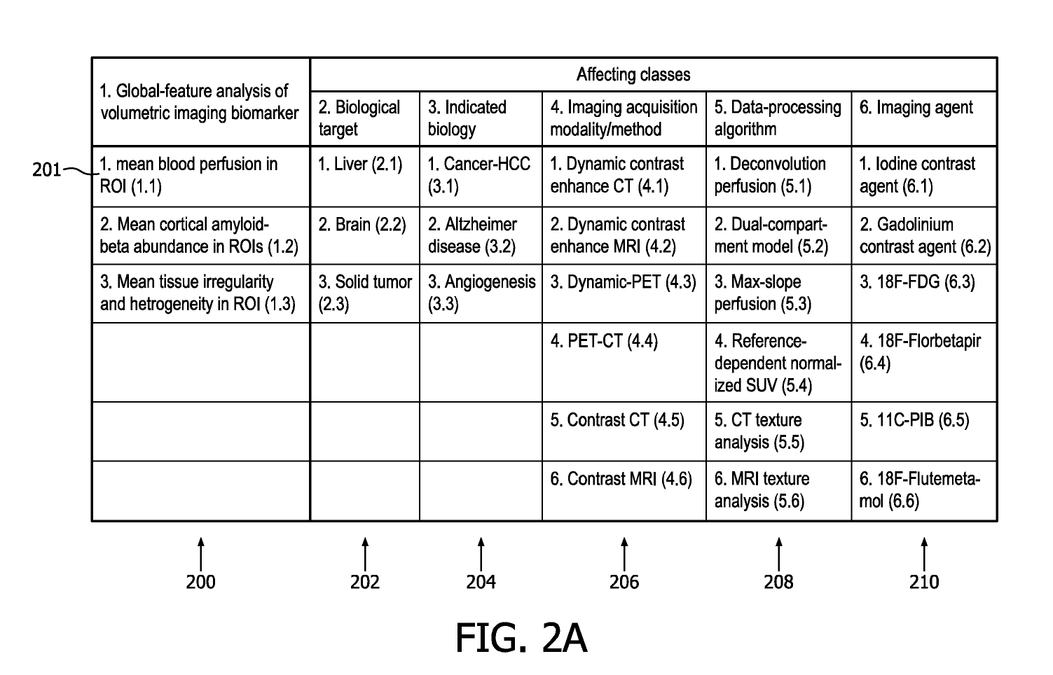

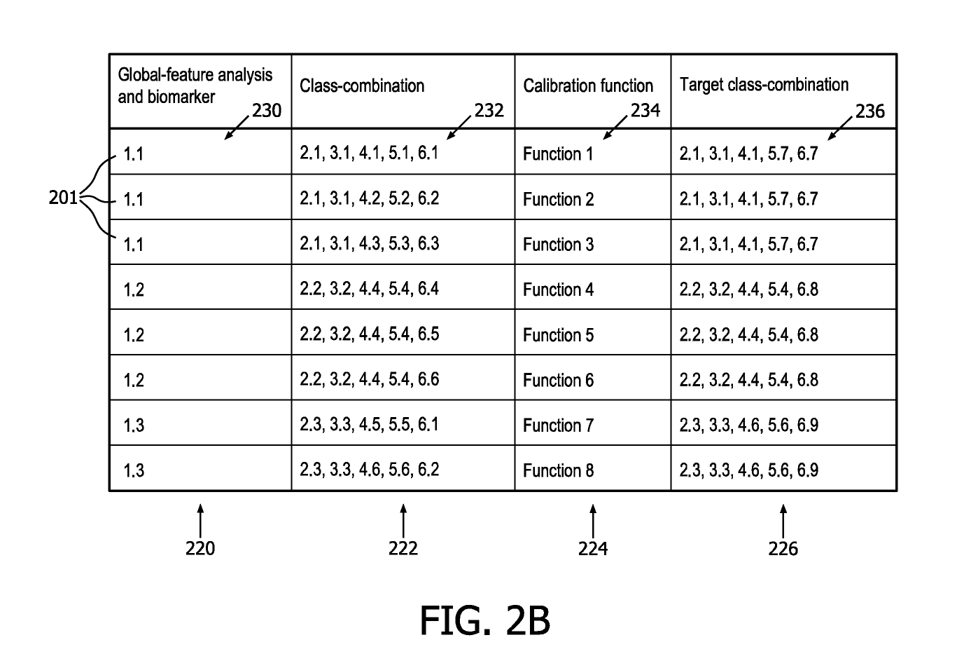

With reference to FIGS. 2A and 2B, partial examples of multi-class calibration data from the multi-class calibration database 108 is illustrated. In FIG. 2A, the multi-class calibration database 108 is shown to include a list of global features analysis 200, such as mean blood perfusion (1.1), mean cortical amyloid-beta abundance (1.2), and mean tissue irregularity and heterogeneity (1.3). The list of global features analysis 200 are analysis that can be presented by the global feature analyzer 106. For example, a mean blood perfusion analysis 201 (1.1), is selected. A list of biological targets 202 is shown, such as liver (2.1), brain (2.2), and solid tumor (2.3). The list can include organs, tissues, segmented structures, regions of interest, and the like. A list of indicated biology 204 is shown. The indicated biology includes the disease, biological function, and/or biological mechanism to which the analysis is directed, such as cancer (3.1), Alzheimer disease (3.2), and angiogenesis (3.3). A list of imaging acquisition modalities/protocols 206 is shown, such as dynamic contrast enhanced CT (4.1), dynamic contrast enhanced MM (4.2), dynamic PET (4.3), etc. A list of data processing algorithms or techniques 208 is shown, such as deconvolution perfusion (5.1), dual-compartment model (5.2), max-slope perfusion (5.3), reference dependent normalized SUV (5.4), etc. A list of imaging or contrast agents 210 is shown, such as iodine (6.1), gadolinium (6.2), ¹⁸F-FDG (6.3), ¹⁸F-Florbetapir (6.4), etc. Each list is an independent list.

With reference to FIG. 2B, multi-class calibration data is shown which includes a list of global feature analysis 220 related by class-combinations 222 to calibration functions 224. Each global feature analysis 230 can be related to one or more calibration functions 234 by one class-combination 232 for one target-class combination 236. That is one class-combination 232 can include two different functions, e.g. two different calibrations and each for a different target class-combination. Each class-combination 232 defines a valid function 234, e.g. function exists which transforms pre-calibrated quantitative measurements to calibrated quantitative measurements.

For example, for a mean blood perfusion (1.1), a class-combination of a liver biological target (2.1), an indicated biology of cancer (3.1), an imaging modality/method of dynamic contrast enhanced CT (4.1), a data processing algorithm of deconvolution perfusion (5.1), using an iodine (6.1) contrast agent, uses a function 234 to transform the pre-calibrated mean blood perfusion measured with the class-combination to a calibrated mean blood perfusion. The calibration function can include a target class-combination 226 or description, which indicates the characteristics of the calibration.

In one instance, one of the target class-combinations 226 can represent a gold-standard class-combination and the calibration function 224 transforms the pre-calibrated measures to the calibrate measures, e.g. measures as if the patient was imaged and analyzed using the gold-standard class-combination. In another instance, with no such gold standard existing, and multiple class-combinations that exist, the calibration function 224 of the target class-combination 226 represents an average of the multiple class-combinations.

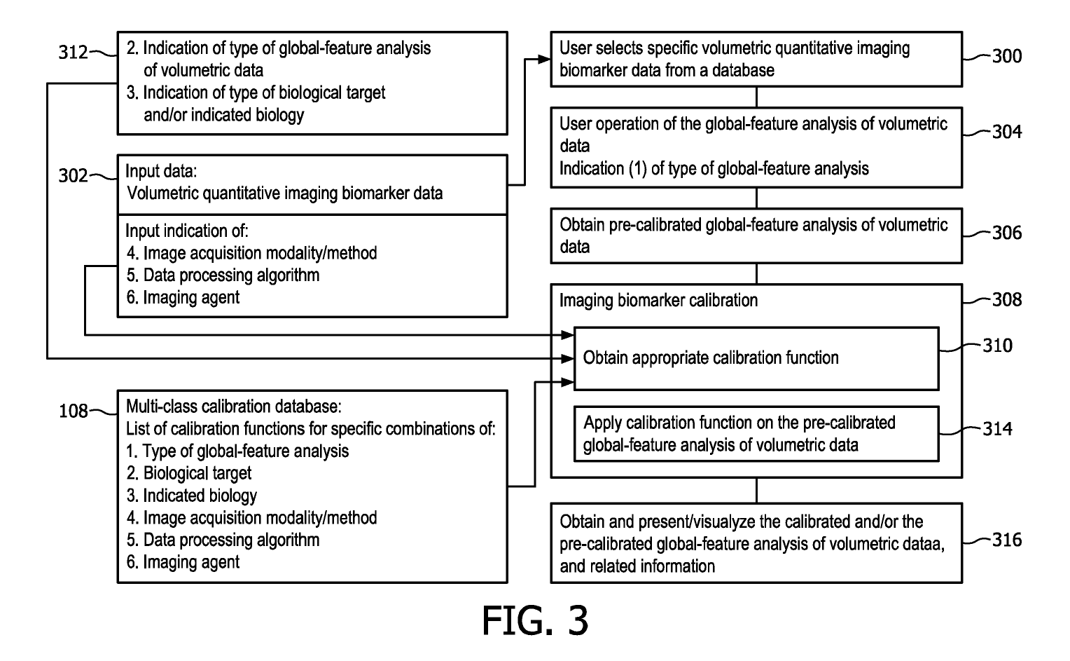

With reference to FIG. 3, an example method of cross procedure quantitative biomarker imaging is flowcharted. At 300, imaging data 302 including biomarker data is received. The imaging data is received from computer memory or storage, such as the PACS 104, or from the one or more imaging devices 102. The imaging data 302 includes volumetric quantitative biomarker data, e.g. contrasted volumetric data. The imaging data 302, e.g. DICOM metadata and/or separate input can include the acquisition modality and/or protocol, a data processing technique, and/or a contrast agent.

At 304, the selection of a global features analysis 230 is received. For example, mean blood perfusion is received. Receiving the selection can include retrieval of the list of global features analysis 200 from the multi-class calibration database 108 and presentation on the display device 110. Retrieval and/or presentation can include refinement of the list of global features analysis 200, e.g. keyword retrieval using manual input and/or DICOM metadata and/or reduction in presentation based on the manual input and/or the metadata.

At 306, the received imaging data is analyzed using the tools 114 to generate one or more quantified measurements. For example, a blood flow measurement tool is used to generate a mean value in a defined volume of blood perfusion in units of rate computed from a CT contrast enhanced image time series.

At 308, using the selected global features analysis and the multi-class calibration database 108, the one or more pre-calibrated measurements are transformed to calibrated measurements. The calibration function 234 is retrieved from the multi-class calibration database 108 based on the selected 230 global-feature analysis and the class-combination 232 at 310. The class-combination can be determined from other input 312, such as selection from the list of the biological targets 202 and the list of the indicated biology 204, and/or the imaging data 302. At 314, the retrieved calibration function 234 is applied to the non-calibrated measurements to transform the pre-calibrated measurements to the calibrated measurements.

At 316, the calibrated measurement is displayed on the display device 110 and/or stored in the PACS 104. The pre-calibrated measurement can be displayed on the display device 110. The calibrated measurement and/or the pre-calibrated measurement can be visualized as numerical values, and/or graphically.

The above may be implemented by way of computer readable instructions, encoded or embedded on computer readable storage medium, which, when executed by a computer processor(s), cause the processor(s) to carry out the described acts. Additionally or alternatively, at least one of the computer readable instructions is carried by a signal, carrier wave or other transitory medium.

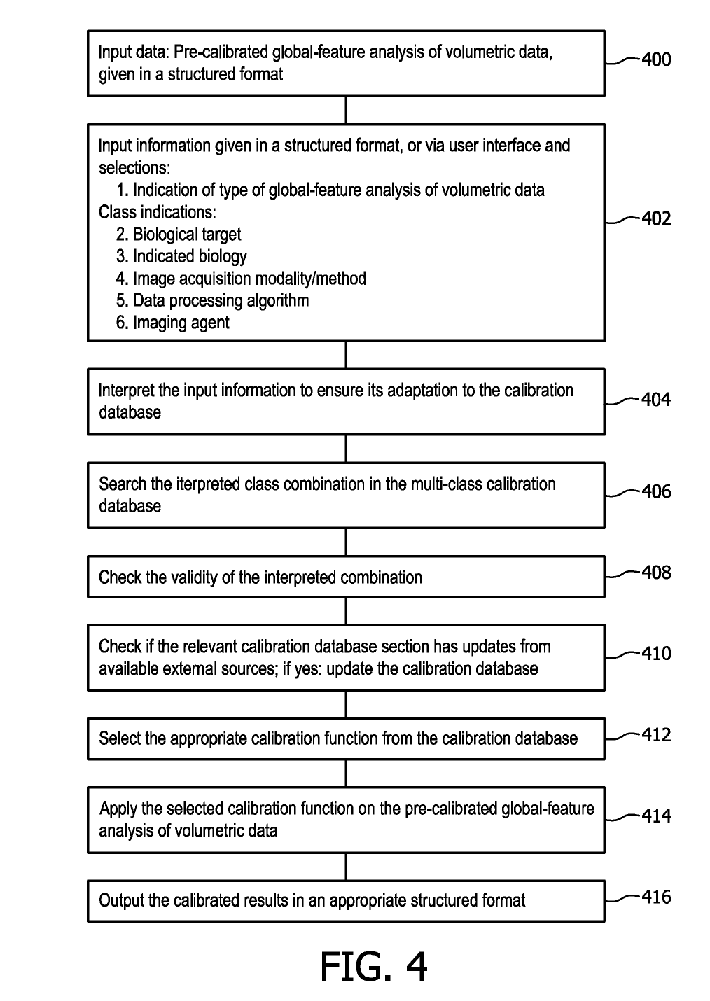

With reference to FIG. 4, another example method of cross procedure quantitative biomarker imaging is flowcharted. At 400, one or more pre-calibrated measurements are received. For example, pre-calibrated mean concentrations of a contrast agent in a first and a second anatomical structure, and a pre-calibrated maximum concentration in the first and the second anatomical structure are received.

At 402, the global features analysis 230 and inputs for each of the classes are received. Inputs for each of the classes can include words that are input from the input device 112.

At 404, the indications, such as input data or signals are interpreted to match each of the indications to the multi-class calibration database 108. For example, each input is compared to one of the class lists as described in reference to FIG. 2A to determine if the input is present in the class list. The interpretation can use an ontological dictionary to match the input to the words used in each class list.

At 406, the multi-class calibration database 108 is searched using the interpreted class-combination to locate the class-combination. Example class-combinations are described in reference to FIG. 2B.

At 408, the searched class-combination is checked for validity. If the interpreted class-combination is located in the multi-class calibration database 108, the located interpreted class-combination can be displayed on the display device 110 and an input indication signal or data received indicating confirmation. If the interpreted class-combination is not located in the multi-class calibration database 108, similar class-combinations can be displayed for manual selection of an alternative class-combination.

The multi-class calibration database 108 can be checked against external sources for updates at 410. The updates can be according to class-combinations. The updates can be accessed and updated.

At 412, the calibration function is selected based on the class-combination. The calibration function can be selected from one or more target class-combinations. The quantitative imaging biomarker calibrator 116 loads the selected calibration function.

The quantitative imaging biomarker calibrator 116 transforms the one or more pre-calibrated measurements to calibration measurements by applying the selected calibration function at 414. Continuing the above example at 400, the pre-calibrated mean concentrations of the contrast agent in the first and the second anatomical structure, and the pre-calibrated maximum concentration in the first and the second anatomical structure are transformed to calibrated mean concentrations of a second contrast agent in the first and the second anatomical structure, and calibrated maximum concentration in the first and the second anatomical structure.

At 416, the calibrated measurements are output, e.g. to the display device 110 and/or the PACS 104. The output can include the pre-calibrated measurements and the calibrated measurements. The output can include a structured format. The output can include text/numerical formats and/or graphical formats.

The above may be implemented by way of computer readable instructions, encoded or embedded on computer readable storage medium, which, when executed by a computer processor(s), cause the processor(s) to carry out the described acts. Additionally or alternatively, at least one of the computer readable instructions is carried by a signal, carrier wave or other transitory medium.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A quantitative measurement system, comprising:
a quantitative imaging biomarker calibrator configured to receive one or more pre-calibrated quantitative measurements of imaging data obtained according to a global features analysis and a class-combination of a biological target, an indicated biology, an imaging acquisition modality and a protocol, a data processing technique, and a contrast agent, and apply an identified function to the one or more pre-calibrated quantitative measurements to compute the one or more calibrated quantitative measurements based on a target class-combination which is different from the class-combination.

2. The system of claim 1, further comprising:
a multi-class calibration database which includes a plurality of global feature analysis and class combinations of classes of biological targets, indicated biologies, imaging acquisition modalities and protocols, data processing techniques, and contrast agents, and each class combination includes at least one function; and
a global feature analyzer configured to receive the global features analysis for the imaging data and input to determine the class-combination to identify the function in the multi-class calibration database.

3. The system according to claim 1, further including:
a display device configured to display the one or more quantitative measurements and the one or more calibrated quantitative measurements.

4. The system according to claim 1, wherein the global features analyzer is configured to input meta-data of the imaging data to determine one or more classes of the class-combination.

5. The system according to claim 1, wherein the global feature analyzer is configured to compare inputs received from an input device with instances in one or more of the classes.

6. The system according to claim 5, wherein the global feature analyzer is configured to compare the inputs with the instances in one or more of the classes using an ontological dictionary.

7. The system according to claim 1, wherein the quantitative imaging biomarker calibrator is configured to apply the identified function, which is a linear function of the pre-calibrated quantitative measurements.

8. The system according to claim 1, wherein the quantitative imaging biomarker calibrator is configured to apply the identified function, which is a non-linear function of the pre-calibrated quantitative measurements.

9. The system according to claim 1, wherein the global features analyzer is configured to search the multi-class calibration database and determine the validity of the class-combination.

10. The system according to claim 1, wherein the global features analyzer is configured to receive a signal indicative of a selection of the applied identified function from a plurality of functions having a same class-combination in the multi-class calibration database.

11. A method of quantitative measurement, comprising:
applying an identified function to one or more pre-calibrated quantitative measurements obtained according to a global features analysis and a class-combination of a biological target, an indicated biology, an imaging acquisition modality and a protocol, a data processing technique, and a contrast agent to compute the one or more calibrated quantitative measurements based on a target class-combination which is different from the class-combination.

12. The method according to claim 11, further comprising:
receiving the global features analysis for the imaging data and input to determine the class-combination which identifies the function in a multi-class calibration database which includes a plurality of global feature analysis and class combinations of classes of biological targets, indicated biologies, imaging acquisition modalities and protocols, data processing techniques, and contrast agents, and each class combination includes at least one function.

13. The method according to claim 11, further comprising:
displaying the one or more quantitative measurements and the one or more calibrated quantitative measurements.

14. The method according to claim 1, wherein receiving the global features analysis for the imaging data and input to determine the class-combination includes inputting meta-data of the imaging data to determined one or more classes of the class-combination.

15. The method according to claim 11, wherein receiving the global features analysis for the imaging data and input to determine the class-combination includes comparing inputs received from an input device with instances in one or more of the classes.

16. The method according to claim 15, wherein comparing inputs includes using an ontological dictionary to interpret and relate the inputs to the instances in one or more of the classes.

17. The method according to claim 11, wherein applying includes applying the identified function, which is a linear function of the pre-calibrated quantitative measurements.

18. The method according to claim 11, wherein applying includes applying the identified function, which is a non-linear function of the pre-calibrated quantitative measurements.

19. The method according to claim 11, further including:
searching the multi-class calibration database and determining the validity of the class-combination.

20. A quantitative measurement system, comprising:
a multi-class calibration database which includes a plurality of global feature analysis and class combinations of a biological target, an indicated biology, an imaging acquisition modality and method, a data processing algorithm, and a contrast agent, and each class combination includes at least one function;
a global feature analyzer configured to receive a selection of a global features analysis for imaging data and class information to identify at least one function in the multi-class calibration database which transforms one or more quantitative measurements of the imaging data to one or more calibrated quantitative measurements; and
a quantitative imaging biomarker calibrator configured to apply the identified at least one function to the one or more quantitative measurements of the imaging data to compute the one or more calibrated quantitative measurements and the identified at least one function of the identified class combination transforms the one or more quantitative measurements of the imaging data to the one or more calibrated quantified measurements of a target class combination and the target class combination is different from the identified class combination.
